(19) United States
(12) Patent Application Publication
(10) Pub. No.: US 2013/0338697 A1
(43) Pub. Date: Dec. 19, 2013

(54) DILATION DEVICE

(71) Applicant: Gianni Leoni, Sachseln (DK)

(72) Inventor: Gianni Leoni, Sachseln (DK)

(21) Appl. No.: 14/002,819

(22) PCT Filed: Mar. 2, 2012
(86) PCT No.: PCT/EP2012/000930

§ 371 Date: Sep. 3, 2013

(30) Foreign Application Priority Data
Mar. 5, 2011 (DK) PA 2011 00151

(51) Int. Cl. A61M 29/02

(52) U.S. Cl. CPC A61M 29/02
USPC 606/192

(57) ABSTRACT

Dilation system comprising a dilatable device made from a thermoplastic material having a closed end and an open end, the open end being connected to a pressure source, said dilatable device being able to be inflated by the application of pressure and deflated by loss of pressure to retain almost its original dimension, wherein the dilatable device has at least two layers (121, 161) of said thermoplastic material and sandwiched in between said two layers (121, 161) of thermoplastic material a braided mesh (41) made from a multitude of metal wires, wherein the braided mesh (41) of the inflatable device in the non-pressurized state has a braiding angle (γ) of less than 54.7° in at least one portion of the inflatable device and a braiding angle (α) of more than 54.7° in at least one other portion of the inflatable device such that under internal pressure the braiding angle (γ) in the at least one portion of the inflatable device will increase to an angle (δ) under increase of diameter and reduction of length and the braiding angle (α) in the at least one other portion of the inflatable device will decrease to an angle (β) under reduction of diameter and increase of length, the length increase in the at least one other portion compensating for the length reduction in the at least one portion.

### TECHNICAL FIELD

The present invention relates to a dilation system comprising a dilatable device made from a thermoplastic material having a closed end and an open end, the open end being connected to a pressure source, said dilatable device being able to be inflated by the application of pressure and deflated by loss of pressure to retain almost its original dimension.

Dilation systems and in particular dilation balloons are frequently used in the medical field, e.g. in endovascular applications for coronary angioplasty, also known as PTCA.

Such dilation balloons have two characteristics, which may be considered as disadvantages. One such a property is a tendency to not go back to the original dimensions after inflation. This is valid for both test inflations after production and before application with a patient and after inflation in a patient's body. The dimension of such a balloon before inflation and after inflation differ a little bit in volume diameter and length. After deflation, the cross section of the balloon is greater than the cross section before inflation, and the length is diminished. After use with the patient, such deflated balloon may cause trauma in a patient's vessel by the so-called wing effect.

The second drawback is the usual displacement during application and inflation. With a conventional balloon, an increase of diameter is connected to a reduction of lengths. The shortening of the balloon length results in a relocation of the balloon tip in direction of the proximal end of the balloon, and at the same time a lateral movement of the balloon surface relative to the vessel wall. This results in a loss of precision of the dilation treatment.

In view of this, it is an object of the invention to provide a dilation system, which takes account of these observations and in particular allows a dilation device to regain its original dimension, as far as possible, after deflation and does not undergo the length reduction upon inflation.

These objects are met with a dilation system, as described in the introductory part, wherein the dilatable device is able to be inflated by the application of pressure and deflated by loss of pressure to retain almost its original dimension, wherein the dilatable device has at least two layers of said thermoplastic material and sandwiched in between said two layers of thermoplastic material a braided mesh made from a multitude of metal wires, wherein the braided mesh of the inflatable device in the non-pressurized state has a braiding angle (γ) of less than 54.7° in at least one portion of the inflatable device and a braiding angle (α) of more than 54.7° in at least one other portion of the inflatable device such that under internal pressure the braiding angle (γ) in the at least one portion of the inflatable device will increase to an angle (δ) under increase of diameter and reduction of length and the braiding angle (α) in the at least one other portion of the inflatable device will decrease to an angle (β) under reduction of diameter and increase of length, the length increase in the at least one other portion compensating for the length reduction in the at least one portion.

Preferred embodiments of the dilation system according to the invention are described in the subclaims. In particular, the at least one portion of the dilatable device is a proximal portion and the at least one other portion is the distal portion. However, there may be more than only one portion and more than only one other portion.

Preferably, the wires of the braided mesh have a tensile strength of at least 20 cN and a diameter of 10 to 100 μm. Preferred is an embodiment, where the metal wires are steel wires with a diameter of about 20 μm.

The preferred material for the thermoplastic layers is polyurethane, especially one with a Shore hardness of at least 50 A. The thermoplastic polyurethane material should follow Hook's Law.

Conventionally, the device has a central guide wire lumen, where the lumen is made from a thermoplastic material having a reinforcing metal wire coil to prevent the kinking of the device. The reinforcing metal wire coil should be made from a wire band with ragdangular cross section covering the thermoplastic material of the lumen, or being covered thereby.

The dilation system according to the invention most preferably is a balloon catheter.

The present invention relates to the unique system of dilation properties to be able to impede the longitudinal movement of the enlargement closed section due to internal pressure of a reinforced thermoplastic braided tube with mono or multiple filaments placed between two or more thermoplastic layers forming a tubular thernnoplastic metal wire reinforced sandwich and having one open proximal end for pressure application comprising a shorter elongated section for negative expansion. The compensation physical factor is a system of balancing the movement of the acting forces in the X-Y plain having the characteristics that in the X-axis direction the displacement must counter balance the displacement in the Y-axis in positive direction which displacement is negative in the X-axis direction being a continuous part of an elongated section longer than the expansion section having a close distal end to be able to change the original dimension by having in the X-Y plain a positive displacement in the Y-axis greater than the negative displacement of the X-axis and counteract this movement of the positive displacement in the Y-axis by the elongated section during the application of the internal pressure resulting in radial and axial forces being in opposite direction, see Graph 1,2. This compensation physical factor, Fₓ and F_{y} and F_{z} being a function of Fₓ and F_{y} forces arising from the internal pressure, is built up by braided metal wire placed in between two layers of thermoplastic Hookian material forming a tubular reinforced sandwich where the metal wire meshes are able to move in different direction under internal pressure, but always keeping the crossing point and changing braided angles and pitches at different sections.

The invention is not only related to a dilation catheter system comprising an open proximal and a second distal closed end, but to any system where the applied internal pressure and the resulting forces are working in opposite direction resulting in a fixed distal point position (Pₒ) eliminating any movement in the X-axis direction of the device under internal pressure. The reinforcement material being placed between an internal and external layer forming a tubular thermoplastic metal wire reinforced sandwich which permits the braided reinforced tube with mono or multiple filament to move in relation to the applied internal pressure always having forces working at the same time in opposite direction, F_{y} and Fₓ. The Fₓ is the positive force causing the displacement of the device in the X-axis direction at the same time causing a negative displacement in the Y-axis direction. The F_{y} is the force that causes the displacement in the Y-axis positive direction and at the same time causes a negative displacement in the X-axis direction. This balance of displacement caused by the F_{y} and Fₓ in the X-Y plain during the applied internal pressure will always allow to maintain the original position independent of the value of the Fₓ and F_{y}. The F_{z} is a function of the two opposite working forces Fₓ and F_{y} forces arising from the internal pressure.

### BACKGROUND ART

A thermoplastic tube exposed to internal stresses causing a continuous deformation of the original dimension will at certain times pass the transition point of modulus of elasticity (E) and not be able any more to recover the original dimension after stress release.

A metal wire reinforced thermoplastic material in a sandwich form by braiding having meshes can prevent the internal stresses to overpass the point of modulus of elasticity (E) even with increased internal stress causing the wire meshes to adjust their angle to a neutral value of 54.7°. This value is independent of the tube size. When the applied internal stress value is below the breaking force of the metal wire, there will not be any more deformation and the thermoplastic sandwich having embodied the metal wire can recover the original dimension after stress relief.

The dilation device can be applied in several areas, industrial (sewer lines), drainage and in the medical sector, enlargement of bones in the spine, balloon dilation catheters for treatment of stenosis in a blood vessel. During the years there have been several other devices used with the main purpose to enlarge the blood vessel without using balloon dilation catheters, i.e. rotor blade, laser, Straub catheter the use of which has nothing in common with a balloon dilation catheter except to reopen the blood vessels for better blood flow. It is very well known to every physician in the field that the use of reopening the blood vessel from stenosis results in a better blood flow. This intervention of dilation is very old, already from the Judkins-Dotter-Gruentzig time in the sixties, which was the beginning of the procedure of dilation of the blood vessel, and with the passing decade more sophisticated device systems have been developed. It is very clear to every person with a basic knowledge in elementary physics that when two opposite forces of equal value apply at the same time and on the same object, there will be no position movement. The question is to find a mechanical system that allows such demand to be reliable and reproduceable. The use of reinforced method in thermoplastic material has been known for many decades, in particular in the area of aerospace, cars, boats, tyres and thousand others. The application of reinforcement in the medical sector has become more developed in the last decade. However, the standard dilation balloon catheters are made of thermoplastic materials only, such as silicone, polyurethane, nylon, and many others and are unable to withstand high pressure or are too rigid for bending. In the reinforcement technique of balloon dilation catheters it is known to comine thermoplastic material with steel wire or other similar materials like NITINOL, PtIr (platinumiridium), W (tungsten), different alloys etc. Braiding machines able to work with thermoplastic mono or multiple filaments down to 20 microns with calibration carrier tensioned as low as 3 cN with different numbers of carriers, are known in the art, one being the LEONI braider.

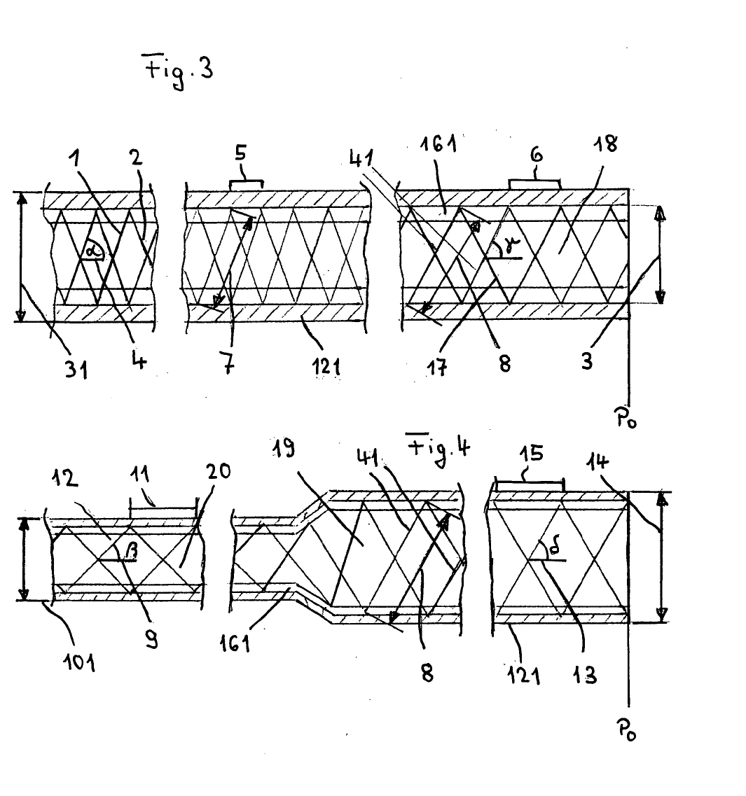

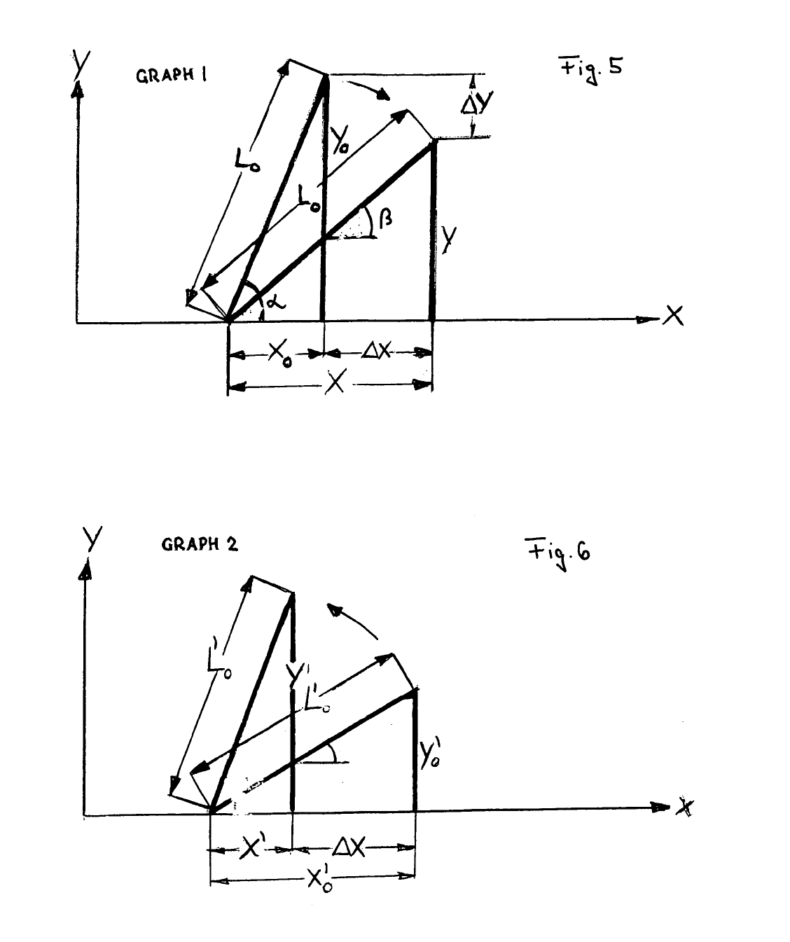

There are several balloon devices with different designs and application methods for enlargement of blood vessels giving a better cross-section of the blood flow. The dilation catheter with a preformed balloon is mainly made from a thermoplastic tube being enlarged to a certain diameter and a certain length by a mechanical system. Each one of the above mentioned devices for enlargement purposes of the blood vessel has advantages and disadvantages with balloon dilation catheters non- or reinforced by mono or multiple filaments which can be metal wire or synthetic material. Preformed thermoplastic balloons non reinforced used in connection with dilation have some disadvantages compared with the reinforced balloons for the same application. These disadvantages are mainly the following: Not being able to accommodate in a bending area of the blood vessel when inflated, long deflation time, risk of rupture in contact with sharp protuberances, tendency to neck formation, wings formation after deflation with a risk of inner surface scrapes. These disadvantages are not present in a system of the present invention having the physical factors Fₓ and F_{y} embodied where the applied internal pressure acting in the metal wire meshes (41) give rise to balance forces acting in opposite directions giving an unchanged distal position (P₀) (FIG. 3, 4).

### Search Analysis

Among many technical constructions of reinforced dilation balloon catheters, is the U.S. Pat. No. 5,772,681, LEONI, describing a reinforced balloon for dilation purposes where in page 1, dilation catheter, technical field, line 10 to 13 the section not containing balloon dilation is described to be “predominantly unexpandable or expandable to a lesser degree than the balloon section”. This statement is completely opposite to the present invention where the action of the internal pressure reduces the outer diameter of the section, not containing the dilation section, from the initial diameter. “The expansion of the balloon section under internal pressure causes a retraction of the original position in the blood vessel.”, again is opposite to the present invention where the (Pₒ) position remains unchanged. According to description of OLBERT (GB 1.566,674.), the Olbert balloon, instead of having a reticular braided metal wire reinforcement uses a thermoplastic mono filament and applies a spiral technique using two opposite directions of winding around a rotating and vertically moving metal wire mandrel dip coated with polyurethane. U.S. Pat. No. 4,706,670, ANDERSEN/LEONI, is mainly intended for PTCA application. This construction is very similar to the Olbert balloon having a rotating horizontal metal wire mandrel between two distal points, being coated with polyurethane filament of two different hardnesses and melting the filament through a die forming a tube. This coated mandrel was then spirally reinforced with thermoplastic monofilament in two opposite directions and a top layer was then applied in the same way as for the first coating. These dilation balloon catheters are built by using a telescope principle. There are several other balloon dilation catheters with reinforced and non reinforced balloons, for instance U.S. Pat. No. 4,195,637. U.S. Pat. No. 4,706,670. DK 154,870 B and EP 388,486. Neither of them has built in the physical factor Fₓ and F_{y} where F_{z} being a function of Fₓ and F_{y}, forces arising from the internal pressure, applied to the mesh result in a movement in opposite direction and “prevent” a displacement of the balloon during inflation from the area to be treated; this is not the case of the above mentioned patents, neither of those is able, after release of the internal working pressure, to recover the original physical dimension. This is also one of the features of the present invention.

The telescopic principle is the most used system for reinforced dilation balloon catheters where the balloon during inflation is allowed to expand in radial direction due to an internal pressure having an internal plastic tube fixed at the distal end of the balloon section or similar items that function as a sliding unit which moves in retraction or negative direction with respect to the X-axis. Furthermore the movement of the balloon during inflation causing displacement in the X-axis is not avoided. This telescopic principle has nothing in common with the physical factor Fₓ and F_{y} having F_{z} being a function of Fₓ and F_{y} forces arising from the internal pressure, where there are forces working in opposite direction during internal pressure as described in this invention. This undesired situation is only solved by the present invention giving the unique support to the physician to disregard the inconvenience of taking into account the positioning (Pₒ) of the balloon section in the blood vessel. There is no doubt that in this new invention there are many factors which contribute to help the physician by having a dilation system without compliance or displacement during the application of the internal pressure. In any device built up on thermoplastic material independently where it is applied it must be well understood that there are three major factors that deserve consideration: A) time dependency, B) temperature dependency, and C) stress dependency, or in other words (P (t, T, s)). Other factors like environment, UV light, ozon (O₃), liquids, solvents etc., and sometimes the choice of the polymer in relation to the sterilization system, the way how the processing of the device is controlled and many other aspects for the safety of the device used in a human body have to be carefully considered.

### DISCLOSURE OF THE INVENTION

This invention is intended to provide a multiple purpose device to be used in the human body where there are signs of disease(s) to be treated, mainly in the blood vessel system or organs. This new invention of working forces at the same time in opposite direction during internal pressure allows to build devices with many physical characteristics always having embodied the principle of Fₓ and F_{y} and having F_{z} being a function of the Fₓ and F_{y} forces arising from the internal pressure. Among the many possibilities the use of reinforcement by braiding in a tubular thermoplastic metal wire reinforced sandwich construction in combination with thermoplastic polymers is without any doubt the necessity of the application or the embodiment of the physical factor of Fₓ and F_{y} time independent, no compliance. The dilation device according to the present invention has multiple possibility of degree of flexibilities, pushabilities, trackabilities and never the less the possibilities to accommodate this invention to the volumetric configuration in the blood vessel or organ without causing consideration of damage during its use.

The use of embodying the physical factor of Fₓ and F_{y} and having F_{z} being a function of the Fₓ and F_{y} forces arising from the internal pressure, in a dilation system of blood vessels allows to have a larger margin of thermoplastic polymer choice from very soft in hardness “like 50 A” together with the combination of the number of reinforcement braided units resulting always in a mesh design.

The embodiment of the physical factor Fₓ and F_{y} and having F_{z} being a function of the Fₓ and F_{y} forces arising from the internal pressure, into the device to be used in the human body is dependent of the design chosen for a particular application always keeping in mind the goal to help the patient to recover from his disease.

This gives a freedom of building devices without restrictions of sizes and shapes within the use in the human body. The physical factor of embodiment of Fₓ and F_{y} can be placed in any section along the device and according to the present invention is independent where it is placed.

The embodiment of the physical factor Fₓ and F_{y} and having F_{z} being a function of the Fₓ and F_{y} forces arising from the internal pressure, in a medical device is intended to be used as enlargement of blood vessel, spinal bones diseases etc. The mechanical work supplied by the external pressurizing device is adsorbed by the physical factor Fₓ and F_{y} multiplied by the change of the angle of braiding embodied in the reinforced metal wire and considering the thermoplastic polymer deformation in the elastic region responds to a complete recovery of the original geometrical dimension almost immediately after release of the internal pressure.

Another figure of the embodiment of the physical factor Fₓ and F_{y} and having F_{z} being a function of Fₓ and F_{y} allows to build devices having a constant inner and outer diameter before and after the application of internal pressure. This very important figure has predominant factor when the dilation catheter or similar device has to be extracted from the blood vessels or organs without any risk of shearing forces at the wall of the blood vessels damaging the endothelium cells or give rise to the condition in which one or more embolus becomes lodged in an artery and obstructs its blood flow, i.e. embolism.

In accordance with the present invention the reinforced material to be used with physical factor embodied Fₓ and F_{y}, and having F_{z} being a function of the Fₓ and F_{y} forces arising from the internal pressure, during braiding must have physical property respecting the Hook's Law.

The thermoplastic polymer to be used in combination with the physical factor Fₓ and F_{y} embodied in the reinforced material during braiding does not have any possibility for the meshes (41) to react physically or chemically between the thermoplastic layers.

The thermoplastic material to be used in combination with the physical factor Fₓ and F_{y} embodied to the reinforcement material during braiding must have the chemical and physical properties to be accepted for internal contact with the blood vessel or other places in the human body without causing any unwanted reaction where the medical grade is compulsory. In case of industrial application such criteria is not necessary.

In accordance with the present invention where the physical factor Fₓ and F_{y} and having F_{z} being a function of the Fₓ and F_{y} forces arising from the internal pressure, embodied during braiding can have different reinforced materials within the same braiding process. In relation to the invention having a physical factor Fₓ and F_{y} and having F_{z} being a function of the Fₓ and F_{y} forces arising from the internal pressure, embodied by braiding technique with the use of mono or multiple metal wire filaments calibrated in equal tension and displaced in between two or more layers of thermoplastic materials with two or more different angles where during the application of external mechanical work or pressure to the system it is able to accommodate itself resulting in a stable previously positioned area and to be able to adsorb the mechanical work delivered by the external source inflation device, pumps or similar, and delivering the same mechanical work when the external sources, pressure, forces are removed.

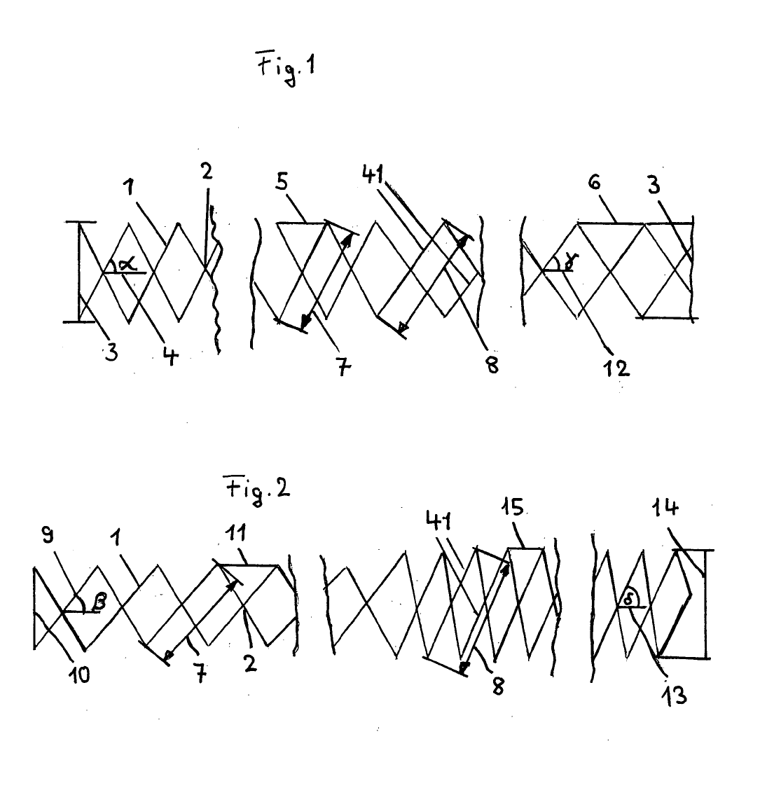

The application of the physical factor Fₓ and F_{y} and having F_{z} being a function of Fₓ and F_{y} forces arising from the internal pressure, by the braiding technique, the mono or multiple braided wire material (1) forming meshes (41) having pre-determined angles (α) (4) in (FIG. 1) and (γ) (12) in (FIG. 1) for each section of the dilation system will during application or external forces from inflation devices, syringes, pumps or similar adsorb mechanical energy. The forces applied to the metal wire meshes incorporated in between two or more layers of thermoplastic material will cause a change in the angles from (α) to (β) (9) in (FIG. 2) and from (γ) to (δ) (13) in (FIG. 2) keeping the point of rotation (2) (FIGS. 1 and 2) of the crossing filament on top of each other constant and independent of changes of the predetermined braided angles. Each section of the dilation system has a preset Angle of the reinforced filament. The different angle of the reinforced filament during application of external forces will give rise to two opposite forces resulting in zero displacement of the distant point (P₀) of the device.

Physical consideration about application of external forces on the volume of the fluid inside the reinforced tubular thermoplastic metal wire reinforced sandwich by braiding. Let P, P′ be the pressure at left and right of the volume element. The fluid at left produces a force Fₗ=P·π·D (Fₗ longitudinal shaft section), the fluid at the right produces a Fᵣ=P′·π·D′ (Fᵣ radial balloon section), dF=−P′·π·D′+P·π·D=—−π(P′·D′−P·D). The P′−P is the pressure at difference between the two sections (shaft−balloon) which has direct influence on the displacement on X−Y axis, let P′−P=dP and π(D′−D)=d_{x,y}.

dF_{xy}=−d(P′−P)·Δ(D′−D)π

When −(P′−P)=0 we have F_{xy}=0

When the internal pressure inside the tubular thermoplastic metal wire reinforced sandwich becomes constant, i.e. P′−P=0, the resultant forces on the X-Y axis are equal and opposite giving rise to unchanged displacement in the two directions, X- and Y-axis.

From the below equation it is possible to calculate the burst pressure or the desired physical properties and in relation to working pressure.

P=burst pressure in Newton (N)/mm²
N₀=numbers of wires
D₀=diameter of final radial expansion (mm)
S=pitch of the wire braided (mm)
α=the angle of the wire to the X-axis
F=breaking force of the chosen braided wire (N)
Pₚ=production value of the burst pressure, Nmm²
Pₗ=theoretical value of burst pressure, Nmm²
K=production factor 0<K<1

### Theoretical Equation:

$${P}_{t} = \frac{2 \cdot No \cdot sin α \cdot F}{S \cdot D} ( N \text{/} {mm}^{2} )$$

$${P}_{p} = {P}_{t} \cdot K 0 < K < 1 K defined by trial \text{-} error technique$$

GRAPH 1—describes the effect of internal pressure on the shaft section having an angle above 55° and resulting in an angle (β), (Y−Y₀)<0. See attached GRAPH 1.

Y=Y₀(sin β)/(sin α)X=X₀(cos β)(cos α)

Known parameters values: L₀; X_{0; α}
L₀=length of the reinforced wire
Y₀=π·D₀ D₀=outer diameter of the tubular thermoplastic metal wire reinforced sandwich

| | |
|---|---|
| Y = π · D′ | D′ = outer diameter with internal pressure |
| (Y − Y₀) = K, K < 0 | negative Y-axis |
| (X₀ + ΔX) = pitch > 0 | positive X-axis |

Positive displacement in the X-axis and negative displacement in the Y-axis.

GRAPH 2—describes the effect of internal pressure on the expanded section having an angle (γ) (FIG. 1) below 55° and resulting in an angle (δ) (FIG. 2) and (Y′−Y₀′)>0. See attached GRAPH 2.

Known parameters: Y₀′; X₀′; L₀′ (length of reinforced wire)

| | | |
|---|---|---|
| Tag γ = X′₀/Y′₀ | Y′₀ = π · D₀ | D₀ = outer diameter of the thermoplastic |
| | | metal |

wire reinforced sandwich
Y′=π·D D=outer diameter with internal pressure

One essential factor to this invention is to obtain a complete and effective result of the physical factor Fₓ and F_{y}, and having F_{z} being a function of Fₓ and F_{y} forces arising from the internal pressure, combining the chosen thermoplastic material to have mechanical characteristics where the modulus of elasticity is in direct relation to the movement of the reinforced braided material during application of internal pressure without preventing the retraction to the original distal position (P0). of the tubular thermoplastic metal wire reinforced sandwich.

Symbol numbers of description in (FIGS. 1, 2, 3 and 4):

| | |
|---|---|
| 1: Reinforced wire | 121: Outer polymer layer |
| 2: Point of angle rotation of the wire | 13: Angle δ, (FIG. 2) |
| 3: Inner diameter of the braiding wire | 14: Outer diameter, (FIG. 4) |
| 4: Angle α | 15: Pitch X′, Graph 2 |
| 5: Pitch (X₀), Graph 1 | 161: Inner polymer layer |
| 6: Pitch (X₀′), Graph 2 | 17: Angle (γ), (FIG. 3), |
| | (12) (FIG. 1) |
| 7: Reinforced wire length (L₀) | 18: Inner diameter, (FIG. 3) |
| 8: Reinforced wire length (L₀′) | 19: Inner diameter, (FIG. 4) |
| 9: Angle (β), (FIG. 2) | 20: Inner diameter, (FIG. 4) |
| 101: Outer diameter, (FIG. 4) | 41: Mesh |
| 11: Pitch (X), Graph 1 | 31: Outer diameter, (FIG. 3) |

### Short Description of Graphs

The representation of the graphs 1 and 2 is present in (FIG. 1) as a symbolic view of the reinforced material placed in a longitudinal dissection without the thermoplastic support. The reinforced braided metal wire (1) forming the mesh (41) has at each pitch a contact point (2) which is the point where the single braided length (7) and (8) for each pitch will remain constant as long as there are no external forces applied internally. Each pitch (5) is disposed through the desired length to have the physical factor Fₓ and F_{y} for stabilization of the X−Y axial displacement. The pitch (6) is another disposition of the braided material having the effect to expand to a larger outer diameter (14) (FIG. 4) when the external applied force is transmitted internally, see (FIG. 2). The change of the angle (α) pos. (4) (FIG. 1) was previously determined by programming the braider, when the internal pressure is applied it forms an angle (β) pos. (9) (FIG. 2) bigger than (α) resulting in a smaller diameter (101) from the previous (31) (FIG. 3, 4). By balancing the displacement value in Graph 2 (Y′−Y₀′)>0 with the displacement value (Y−Y₀)<0 Graph 1. Calculating the difference arising from the two displacements we obtain

G₁=Σ(Y−Y₀)→ΣΔX must be equal to G₂Σ(Y′−Y₀′)→τΔX

### Where |G₁|−|G₂|=0

### G₁ and G₂=Graph 1 and 2

### SHORT DESCRIPTION OF DRAWINGS

The (FIG. 1) represents an open longitudinal cut of the reinforced wire, where the different dimensions of the length of the wire (7) to (8) will remain constant and their different angles (4) to (12) will change their value when internal forces are applied. In this drawing is not present the thermoplastic polymer. The distal section of (FIG. 1) represents a short area of wire braided with a length (8) and with an angle □ (12). This section will during internal pressure modify the outer diameter represented in the distal section (14) (FIG. 2) having the same length of the wire (8), but positioned with an angle δ (13). This change of angles from □ (12) (FIG. 1) to angle δ (13) (FIG. 2) will cause an increase of the outer diameter of the distal section from pitch (6) to pitch (15). The pitch (6) (FIG. 1) represents a preset value before internal pressure application. The pitch (15) (FIG. 2) represents the final value after pressure application. Pitch (5) with angle α will change to pitch (11) with an angle β (9) which means that the outer diameter of this section will decrease. Both of those changes are caused by the internal pressure which is to be found on the physical factor Fx and Fy, forces that are applied to the meshes (41) causing a change of their angles so that the mechanical work arising for such action modifies the original dimension of the tubular thermoplastic metal wire reinforced sandwich.

### HOW THE NOVELTY (INVENTION) IS BUILT UP

There are several approaches to be chosen for the construction of this new idea of Fₓ and F_{y} and F_{z} being a function of Fₓ and F_{y} forces arising from the internal pressure.

One of the preferred constructions (A) is as follows: To have a solid support to be able to withstand the tension of the reinforced material (wires) (1) without any deformation during the braiding process, the choice of support is rather easy from steel wire, copper wire, aluminium, alloys, plastic rod or other metal wire composites. For all these possibilities of choices of internal support for the braiding process there are several other concerns to be taken into consideration: The material support must be able to keep the physical properties unchanged after the braiding process; no changes of the original nominal diameter; no longitudinal deformation; able to be bent without causing deformation on the binding meshes (41) of the braided wires (1); to be able to be removed after the braiding process without causing distortion of the predetermined pitch (5, 6) of the wire; the removal of the support should not impart any damage to the braiding wires in any single section of the braided item.

The choice of the diameter of the support is very important and must always be in relation to the finished braided product. We have chosen to apply this invention by the use of Cu wire as braided support with the following physical property: OD=0.90 mm, max. elongation at break between 25 and 30%. The Cu wire from the bobbins undergoes a straightening process before surface cleaning. The Cu wire is thoroughly cleaned with CH₃CH₂ OH ethanol by the use of a fluff-free cloth and dried by the evaporation of the alcohol. The Cu wire is wound on a larger spool with paper protection for each layer.

The largest spool with the Cu wire is positioned to have a top coating of thermoplastic material different from the one intended for the preparation of the invention. This coverage of the Cu wire is necessary to prevent particles from copper to remain inside the finished braided product, such contamination is dangerous for the human body and poisonous. In our case the coating of the Cu wire is done by the use of PE (polyethylene), but any other choice of coating can be used as long as it does not react chemically or physically with the next coating. The second layer (161) to be applied on top of the Cu—PE was thermoplastic polyurethane TPU, shore hardness 92 A, with a wall thickness of 0.75 mm. This process is done continuously by crosshead extrusion. The use of TPU allows to have no chemical or physical reaction with the PE layer, also after wire braiding.

The braider used in this invention, developed by Gianni Leoni, has 48 carriers which allow a tension control of ±3 cN on the metal wire (1), together with a CNC system. In any case we have chosen a stainless steel wire AISI 304V with OD of 25 microns from Fort Wayne, USA, wound on small aluminium spools with tension control. The 48 spools are then installed in the 48 carriers and the tension for each steel wire was set to 85 cN. The Cu—PE—TPU was then passed through the central die of the braider which was pre-programmed to balance the expansion and retraction during internal pressure preventing movement of the distal point (Pₒ) with four different pitch placed at different distances along the Cu—PE—TPU. After braiding the Cu—PE—TPU AISI 304V steel wire was set up to be coated by extrusion with the same polymer TPU 92 A forming the final tubular thermoplastic metal wire reinforced sandwich with incorporated the physical factor Fₓ and F_{y} with F_{z} being a function of Fₓ and F_{y} forces arising from the internal pressure.

Another construction (B) of this invention is the use of steel wire as braided support. We have chosen steel wire of 0.40 mm diameter being processed with the same procedure (A) and with the same thermoplastic material used with Cu wire as braided support. In this case the pitch values were different and the steel wire used for braiding was ATSI 304V with OD=0.020 mm with a tension on the wire of 27 cN. The final item had an OD of 0.72 mm. The present description of producing items based on this invention does not preclude the possibility of other application in different fields.

A third trial (C) based on the same process was performed by the use of Cu wire as support, OD=0.30 mm coated with PP (polypropylene), wall thickness 0.025 mm and thermoplastic inner layer of aliphatic Nylon 11, melting point 185° C., the reinforced wire was AISI 304V, OD 0.020 mm, with a tension of 27 cN on a braider of 16 carriers using the same basic process as described on the preferred set-up (A) with different programs. This sample had a final OD of 0.58 mm.

The physical test of these preferred set-ups show that the invention is feasible and allows to produce medical devices or other devices to be applied in the industrial area, for instance in sewer pipes for cleaning, for lifting purposes.

The present invention allows to have an internal metal wire coil not present in the drawings made of a flat steel wire AISI 304V used as a passage way for the guide wire during the intervention in the human body. The use of a flat steel wire coil incorporated inside the circular lumen of this invention and being top-coated with thermoplastic material protects the present invention from kinking during its use and allows the fluid to pass between the external coated flat steel wire coil and the internal area of the invention. Normally this internal thermoplastic coated metal coil has both ends open, one is fixed at the distal expanding end section and the other to the connector as a guide-wire port. The application of the metal coil in the present invention can in certain occasions preclude the use of guide-wire. The insertion of the metal wire coil inside the present invention does not work as a compensation factor for the expansion of the balloon section under internal pressure, neither is it intended to be a compensation device normally used with a standard reinforced balloon dilation catheter. In case the guide-wire is compulsory the metal coated coil can be replaced by a thermoplastic tube operating in the same way as the metal coated coil.

Thus, the invention relates to a dilation system incorporating a physical factor of Fₓ and F_{y} and having F_{z} being a function of Fₓ and F_{y} forces arising from the internal pressure, having an open and closed end of a thermoplastic metal wire reinforced tubular sandwich with the principal goal to prevent the displacement of the distal positioned point (P₀) and being able to recover almost to its previous dimension, (FIG. 3, 4), during the application of external forces to the internal volume (19) and (20) of the thermoplastic tubular metal wire reinforced sandwich (161) and (121) embodied with the reinforcement metal wire (1) with different physical properties by braiding process where the reinforced metal wire (1) covers the total length of the tubular construction (FIG. 3) with different physical properties in each longitudinal section by the use of calibrated wire tension during braiding having the reinforced metal wire (1) crossing each other at a fix point (2) distributed along the X-axis parallel positioned to the centreline (FIG. 3) incorporating the physical factor Fₓ and F_{y} and F_{z} being a function of Fₓ and F_{y}, forces arising from the internal pressure, the internal forces produced by the application of the external pressure inside the free volume (18) (FIG. 3) in the internal tubular section of the thermoplastic metal wire reinforced sandwich containing pre-disposed metal wire meshes (41) having different angles, (4), (17), in (FIG. 3) giving rise to changed angles (9) respectively (13) in (FIG. 4) in relation to the centreline being able to convey the mechanical work received by the external applied pressure to contra balance at the same time the axial movements of the tubular thermoplastic metal wire reinforced sandwich in the X-axis direction and allow the expansion in the Y-axis direction without changes of the originally positioned distal point (P₀) (FIG. 3, 4) and able to recover almost to its previous original physical dimension immediately after the internal forces Fₓ and F_{y} of the metal wire meshes (41) are released, wherein the received mechanical work of the meshes (41) due to internal pressure will modify their original physical dimension pitch and angle, and be able to return the same mechanical work during the release of external pressure so that there is no displacement of the previously positioned distal point (P₀) and recovering almost its original dimension.

Moreover, the invention relates to a dilation system incorporating a physical factor of Fₓ and F_{y} and having F_{z} being a function of Fₓ and F_{y} forces arising from the internal pressure, having an open and closed end of a thermoplastic metal wire reinforced tubular sandwich with the principal goal to prevent the displacement of the distal positioned point (P₀) and being able to recover almost to its previous dimension, (FIG. 3, 4), during the application of external forces to the internal volume (19) and (20) of the thermoplastic tubular metal wire reinforced sandwich (161) and (121) embodied with the reinforcement metal wire (1) with different physical properties by braiding process where the reinforced metal wire (1) covers the total length of the tubular construction (FIG. 3) with different physical properties in each longitudinal section by the use of calibrated wire tension during braiding having the reinforced metal wire (1) crossing each other at a fix point (2) distributed along the X-axis parallel positioned to the centreline (FIG. 3) incorporating the physical factor Fₓ and F_{y} and F_{z} being a function of Fₓ and F_{y}, forces arising from the internal pressure, the internal forces produced by the application of the external pressure inside the free volume (18) (FIG. 3) in the internal tubular section of the thermoplastic metal wire reinforced sandwich containing pre-disposed metal wire meshes (41) having different angles, (4), (17), in (FIG. 3) giving rise to changed angles (9) respectively (13) in (FIG. 4) in relation to the centreline being able to convey the mechanical work received by the external applied pressure to contra balance at the same time the axial movements of the tubular thermoplastic metal wire reinforced sandwich in the X-axis direction and allow the expansion in the Y-axis direction without changes of the originally positioned distal point (P₀) (FIG. 3, 4) and able to recover almost to its previous original physical dimension immediately after the internal forces Fₓ and F_{y} of the metal wire meshes (41) are released, wherein there are metal wire braided meshes (41) positioned with different angles and pitch in respect to the central axis and places in different longitudinal areas by continuous process.

Preferably, the braided wire meshes (41) are placed in between two or more layers of thermoplastic material having physical properties that respect the Hook's law.

According to the invention the internal forces Fₓ and F_{y} caused by internal pressure will modify the original outer dimension in decreasing and expanding movements.

According to a preferred embodiment, inside the tubular thermoplastic metal wire reinforced sandwich is placed a metal wire coil of rectangular cross section coated in its total length by a thermoplastic material. The coated metal wire coil of rectangular cross section prevents kinking during bending.

It is self-understanding that the expanding section or sections of the thermoplastic metal wire (1) reinforced sandwich can be placed in any area along the length of the dilation device.

1. A dilation system comprising a dilatable device made from a thermoplastic material having a closed end and an open end, the open end being connected to a pressure source, the dilatable device adapted to be inflated by the application of pressure and deflated by loss of pressure to retain almost its original dimension, characterized in that the dilatable device includes two layers of the thermoplastic material and sandwiched in between the two layers of thermoplastic material a braided mesh made from a multitude of metal wires, wherein the braided mesh of the inflatable device in the non-pressurized state has a first braiding angle, the first braiding angle being less than 54.7° in a first portion of the inflatable device and a second braiding angle, the second braiding angle being more than 54.7° in a second portion of the inflatable device such that under internal pressure the first braiding angle in the first portion of the inflatable device will increase under increase of diameter and reduction of length and the second braiding angle in the second portion of the inflatable device will decrease under reduction of diameter and increase of length, the length increase in the second portion compensating for the length reduction in the first portion.

2. A dilation system according to claim 1, characterized in that the first portion is the proximal portion of the dilatable device and the second portion is the distal portion of the dilatable device.

3. A dilation system according to claim 1, characterized in that the metal wires of the braided mesh have a tensile strength of at least 20 cN.

4. A dilation system according to claim 1, characterized in that the diameter of the metal wires of the braided mesh is 10 to 100 pm.

5. A dilation system according to claim 4, characterized in that the metal wires are steel wires with a diameter of about 20 pm.

6. A dilation system according to claim 1, characterized in that thermoplastic layers are made from polyurethane having a Shore hardness of at least 50 A.

7. A dilation system of claim 6, characterized in that the thermoplastic polyurethane materials follows Hook's law.

8. A dilation system according to claim 1, characterized in that the dilatable device has a central guide wire lumen, where the lumen is made from a thermoplastic material having a reinforcing metal wire coil to prevent kinking of the device.

9. A dilation system according to claim 8, characterized in that the metal wire coil is made from a wire band with rectangular cross section covering or covered by the thermoplastic material of the lumen.

10. A balloon catheter including the dilation system according to claim 1.
